(19) United States
(12) Patent Application Publication
(10) Pub. No.: US 2005/0143625 A1
(43) Pub. Date: Jun. 30, 2005

(54) Method and device for covering a medical instrument

(76) Inventors: Willet F. Whitmore III, Long Boat Key, FL (US); Stephen E. Brauner, Bradenton, FL (US)

Correspondence Address: MERCHANT & GOULD PC, P.O. BOX 2903, MINNEAPOLIS, MN, 55402-0903, US

(21) Appl. No.: 11/004,042

(22) Filed: Dec. 3, 2004

Related U.S. Application Data
(60) Provisional application No. 60/526,268, filed on Dec. 3, 2003.

(51) Int. Cl. A61B 1/00

(52) U.S. Cl. 600/121; 604/163; 600/171

(57) ABSTRACT

A method is provided for applying a medical instrument cover including: providing a medical instrument comprising an elongate member, the elongate member having a first end configured for insertion into a body of a patient and a second end spaced from the first end; providing a flexible cover having a closed end, an open end, and sidewall defining an inner region between the closed end and open end; covering at least the first end of the elongate member with the flexible cover; sealing the flexible cover and the instrument to each other to form an enclosed space comprising at least the first end of the elongate member; evacuating fluid from within the enclosed space to form an evacuated enclosed space, with the flexible cover conforming to at least a portion of the elongate member within the evacuated enclosed space.

### CROSS-REFERENCE TO RELATED APPLICATIONS

The benefit of Provisional Application No. 60/526,268 filed Dec. 3, 2003 is claimed under 35 U.S.C. § 119(e), and the entire content of this application is expressly incorporated herein by reference thereto.

### FIELD OF THE INVENTION

The invention relates to a method and device for covering a medical instrument. More particularly, the invention relates to a cover for creating an air-tight seal around at least a portion of a medical instrument.

### BACKGROUND OF THE INVENTION

Most invasive medical procedures require a sterile field and sterile instruments. However, some medical instruments cannot be sterilized or may not be sterilized in a practical or cost effective manner. This may be because of the physical characteristics of the materials used to make the instrument, components that cannot tolerate the various methods of sterilization, or because of the mechanical features that make sterilization impractical or impossible without destruction of the part. In these instances, a sterile cover or drape for the chosen instrument will be required. Most commonly a cover supplied for instruments of this type is a pre-sterilized, single use, disposable product that may be made of a bio-compatible plastic film. Such covers typically are dimensioned and shaped to provide adequate protection against contamination for the part of the instrument that lies within the sterile field while permitting satisfactory function of the instrument for its intended use. The covers also must be easy to place over the instrument without user contamination. All of these features are critical.

Many prior art covers placed on instruments have not had a closely conforming fit thereon. In particular, a low tolerance has been selected for the closeness of fit in order to accommodate other variables such as the shape of the instrument, the movement required of the instrument or its components, production costs or other requirements. Therefore, prior art cover products for use with instruments typically have a loose and somewhat sloppy fit. In general, a sub-optimal fit is tolerated and accepted as a trade-off for low cost and ease of placement of the cover with respect to the instrument. Prior art methods for reducing the excess cover material and/or achieving a more conforming fit of a cover include taping and elastic banding, in which excess material of the cover is bunched close to the instrument. Sterile closures for medical equipment, for example, are disclosed in U.S. Pat. No. 5,873,814 to Adair, in which a tie or an adhesive tape is used to create a sterile enclosure.

Closely conforming sterile covers are known in the medical art. For example, U.S. Pat. No. 6,805,453 B2 to Spetzler et al. discloses a microscope on a stand which are at least partially covered by a drape. The air inside the drape is removed by a suction unit, and the drape is fixed to a portion of the stand at a collar. The patent describes a method for operating such instrument in a way that negative effects of the drape on a surgical operation or procedure are minimized.

As shown in FIGS. 1A and 1B, a typical prior art sterile instrument cover 10, 10′ provides a generally loose fit over an instrument 12, 12′, respectively, which may have an elongate and/or irregular shape. In order to secure the cover 10, 10′ on the respective instrument, a length of tape or elastic band 14, 14′ is placed externally of the cover and instrument to provide a coupling force and compress the cover 10, 10′ proximate an end thereof on the instrument 12, 12′, respectively. While a first end 16, 16′ of instrument 12, 12′ may have a close fit with the cover, the remainder of the cover may generally loosely fit on the instrument.

There remains a need for a method and device for covering a medical instrument that permits a user selectable amount of conformity of the cover shape to the instrument shape.

### SUMMARY OF THE INVENTION

The present invention relates to an inexpensive and practical method in conjunction with a simple device that facilitates the preservation of a sterile field while greatly reducing the nuisance that is often caused by excess material typically present in medical instrument covers. In accordance with the present invention, an improved cover may be made by creating an air-tight seal between the instrument and the cover on the inside of the cover and then evacuating sufficient air to achieve the desired conformity of the cover to the instrument. In some embodiments, at least one strip of mastic or a similar manually manipulatable, pliable, adherent, elastic sealant may be attached to and encircle or otherwise surround the instrument at a suitable location, and thus be used to fill a gap between the instrument and the cover. Such strips of mastic also may encircle and adhere to evacuation tubing that optionally may include a valve attached thereto. The tubing may transit the barrier formed by the mastic, and thus may extend through the mastic.

When the cover is placed on an instrument, it may be compressed onto or otherwise contacted with the mastic to achieve an air-tight seal. The user then may evacuate a desired amount of air from inside the drape to achieve a desired degree of form fit that may be optimal for the given medical procedure and handling of the instrument. Alternatively, the cover may be subjected to a constant vacuum to achieve a near perfect form fit. Preferably, the plastic film is formed of a bio-compatible material that is substantially liquid and gas impervious. Various physical characteristics may be chosen for the plastic film of the cover material to suit a desired use, such as thickness, transparency or opacity, elasticity, softness, resistance to tearing or puncture and any special coatings or embedded chemical agents. Such characteristics may be applicable for procedure and instrument specific covers.

The present invention also has application for non-sterile covers that protect patients from cross-contamination and facilitate cleaning between cases. For similarly shaped devices, a clean but not sterile elastic condom is typically used. For devices with complex shapes or significant length a loose fitting cover must be used to enable easy placement and removal. In these circumstances the improved form fitting that may be achieved by removing the air from within the drape may offer a dramatic improvement in functionality.

Thus, the invention relates to a method for applying a medical instrument cover including: providing a medical instrument comprising an elongate member, the elongate member having a first end configured for insertion into a body of a patient and a second end spaced from the first end; providing a flexible cover having a closed end, an open end, and sidewall defining an inner region between the closed end and open end; covering at least the first end of the elongate member with the flexible cover; sealing the flexible cover and the instrument to each other to form an enclosed space comprising at least the first end of the elongate member; evacuating fluid from within the enclosed space to form an evacuated enclosed space, with the flexible cover conforming to at least a portion of the elongate member within the evacuated enclosed space.

The flexible cover and instrument may be sealed together with mastic, or the flexible cover and instrument may be sealed together with an adhesive material disposed therebetween. The fluid may be evacuated from the enclosed space so that the evacuated enclosed space has a substantially smaller volume than the enclosed space. The fluid may be evacuated from the enclosed space so that the flexible cover substantially conforms to at least a portion of the elongate member within the evacuated enclosed space.

The method may further include: disposing a sealing material on the flexible cover within the inner region; contacting the sealing material to the instrument to seal the flexible cover and the instrument to each other. Also, the method may further include: forming a discrete fluid path from the enclosed space to outside the flexible cover; evacuating the fluid through the discrete fluid path to form the evacuated enclosed space. The discrete fluid path may be a tube, and the flexible cover, instrument and tube may be sealed together with mastic sealing material with the tube extending through the mastic sealing material.

The method may include: disposing a sealing material on the instrument; contacting the sealing material to the flexible cover to seal the flexible cover and the instrument to each other. Also, the method may further include: forming a discrete fluid path from the enclosed space to outside the flexible cover; evacuating the fluid through the discrete fluid path to form the evacuated enclosed space. The discrete fluid path may be a tube, and the flexible cover, instrument and tube may be sealed together with mastic sealing material with the tube extending through the mastic sealing material.

In some embodiments, the flexible cover and the instrument may be sealed to each other by applying an at least partially circumferential adhesive element nearer the second end of the elongate member than the first end. The evacuated enclosed space may not be completely free of fluid. The flexible cover and instrument may be sufficiently sealed to each other to form a substantially nonleaking union.

The invention also relates to a method of forming a sterile field around a medical instrument including: providing a medical instrument comprising an elongate member, the elongate member having a first end configured for insertion into a body of a patient and a second end spaced from the first end; providing a flexible drape having a closed end, an open end, and a sidewall defining an inner region between the ends; covering at least the first end of the elongate member with the flexible drape; sealing the flexible drape and the instrument to each other with mastic to form a first enclosed space comprising at least the first end of the elongate member; removing gas from within the first enclosed space to form a second enclosed space smaller than the first enclosed space, with the flexible drape conforming to at least a portion of the elongate member within the second enclosed space.

The method may further include: disposing the mastic on the flexible drape within the inner region; contacting the mastic to the instrument to seal the flexible drape and the instrument to each other. Also, the method may further include: disposing the mastic on the instrument; contacting the mastic to the flexible drape to seal the flexible drape and the instrument to each other.

The method may involve: forming a discrete fluid path from the first enclosed space to outside the flexible drape; evacuating the gas through the discrete fluid path to form the second enclosed space.

The gas may be removed from the first enclosed space so that the second enclosed space has a substantially smaller volume than the first enclosed space. The flexible drape may substantially conform to at least a portion of the elongate member within the second enclosed space. In addition, the flexible drape and instrument may be sufficiently sealed to each other to form a substantially nonleaking union.

Furthermore, the invention relates to a method for applying a medical instrument cover, including: providing a medical instrument comprising an elongate member, the elongate member having a first end configured for insertion into a cavity of a patient and a second end spaced apart from the first end; applying an at least partially circumferential adhesive element nearer the second end of the elongate member than the first end; providing a flexible cover having a closed end and an open end; covering at least the first end of the elongate member with the flexible cover; contacting a portion of the flexible cover and the adhesive element to form an enclosed space comprising at least the first end of the elongate member; evacuating air from within the enclosed space to form an evacuated enclosed space, whereby the flexible cover substantially conforms to at least a portion of the elongate member within the evacuated enclosed space; and sealing the evacuated enclosed space to prevent gas from passing into the evacuated enclosed space.

### BRIEF DESCRIPTION OF THE DRAWINGS

Preferred features of the present invention are disclosed in the accompanying drawings, wherein:

FIGS. 1A-1B show partial cross-sections of a prior art medical instrument cover;

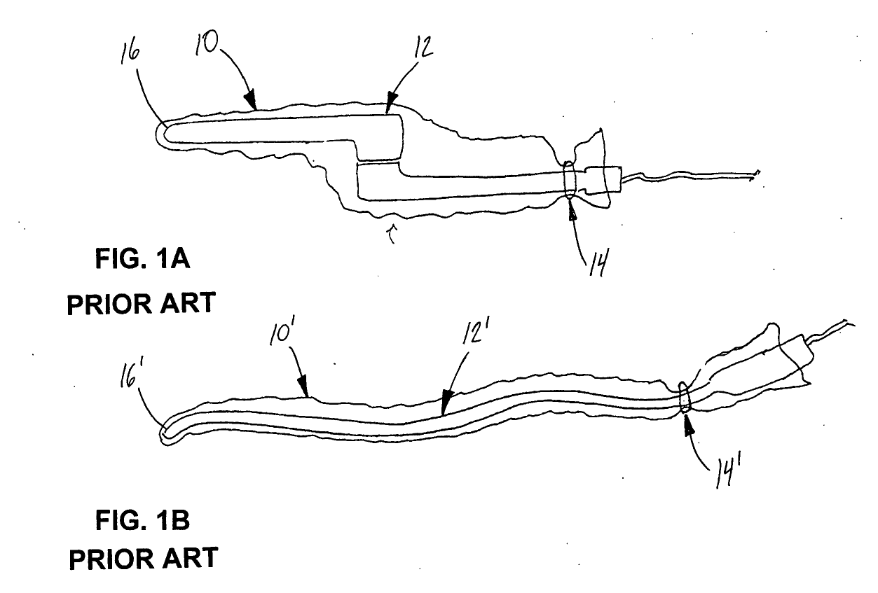

FIGS. 2A-2B show partial cross-sections of a medical instrument cover sealable in accordance with the present invention; and

FIGS. 3A-3C show steps in applying a medical instrument cover in accordance with the present invention, the figures providing partial cross-sectional views.

### DETAILED DESCRIPTION OF THE PREFERRED EMBODIMENTS

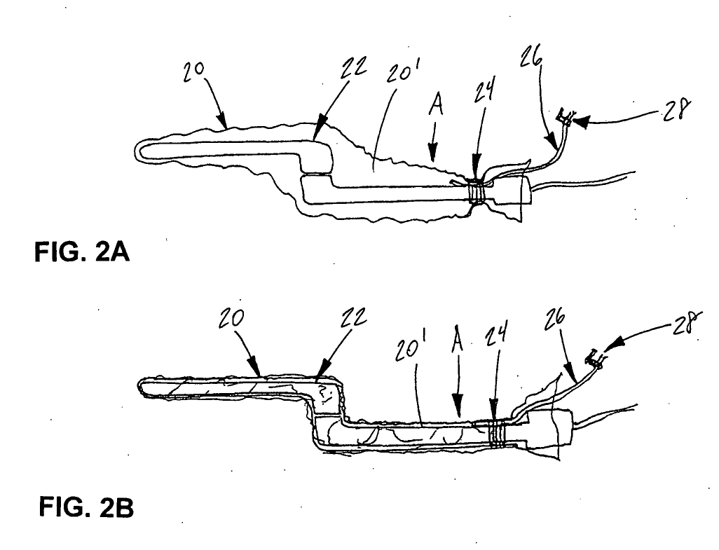

Turning to FIGS. 2A and 2B, a preferred exemplary embodiment of a medical instrument cover 20 and method of use thereof is shown. In particular, cover 20 initially is disposed around instrument 22 as shown in FIG. 2A. A sealant 24 such as a mastic sealant may be applied to one or both of cover 20 and instrument 22, for example proximate an end thereof, so that sealant 24 may act as a bridge to couple and seal space between cover 20 and instrument 22. As nonlimiting examples, sealant 24 for example may be an adhesive putty material or other material with sufficient tactile properties to permit adhesion to cover 20 and instrument 22. Thus, sealant 24 for example may be disposed on the inner surface of cover 20 and subsequently instrument 22 may be contacted with sealant 24, or alternatively sealant 24 may be disposed on outer surface of instrument 22 and subsequently may be contacted with inner surface of cover 20 to form a seal.

Tubing 26 may extend between instrument 22 and cover 20, for example through sealant 24, thus providing a communication path from the inside region 20′ defined by cover 20. Tubing 26 preferably is configured and dimensioned for attachment to a pump such as a vacuum pump, as will be described. A stopcock 28 may be provided proximate a free end of tubing 26, outside of cover 20.

In an alternate arrangement, tubing 26 may extend through a port in, or otherwise be connected to cover 20. For example, tubing 26 may be integrally formed with cover 20. Thus, in some embodiments of the present invention, tubing 20 need not extend through sealant 24, but instead may extend from a side of cover 20 for example proximate region A.

Once a seal is created by sealant 24 so that a substantially nonleaking union is formed between cover 20 and instrument 22, the fluid (gas/air) in region 20′ may be evacuated by attaching a pump to tubing 26. As the fluid is removed from region 20′, cover 20 contracts so that it more closely conforms to the shape of instrument 22. With a substantial amount of such fluid removed from within cover 20, as shown for example in FIG. 2B, the drape 20 surrounds instrument 22 to provide a desired form fit that may be optimal for the given medical procedure and handling of the instrument 22. The amount of conformity may be selected to meet a desired need, and need not exactly match the contours of the instrument 22. Thus, all fluid need not be evacuated from within cover 20. Moreover, the seal created by sealant 24 between cover 20 and instrument 22 need not be “perfect” or free of leaks, but need only be sufficient for fluid to be evacuated from within cover 20 to permit the cover to conform to the shape of instrument 22 as needed.

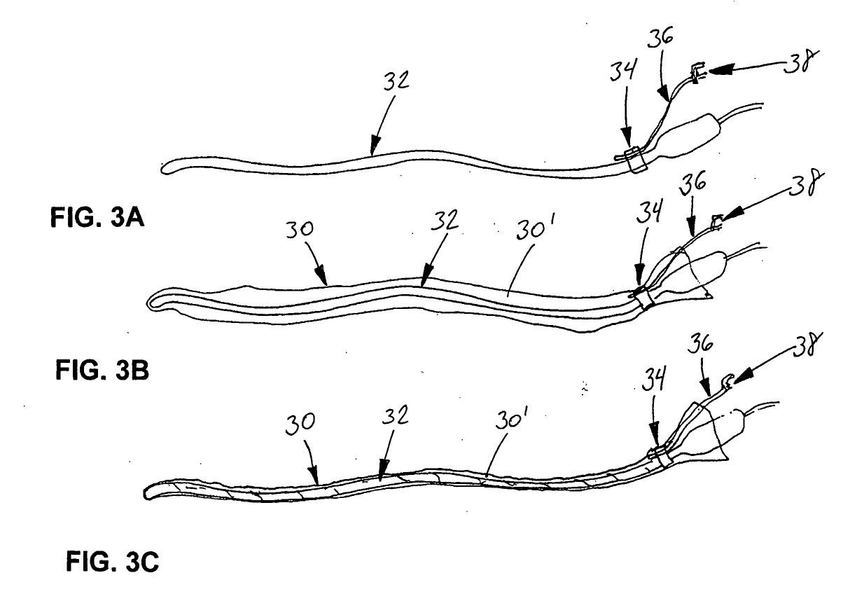

In another exemplary embodiment of the present invention, shown in FIGS. 3A-3C, a drape 30 may be used to cover an instrument 32. In particular, turning to FIG. 3A, a mastic sealant 34 first is applied to instrument 32 with tubing 36 disposed proximate instrument 32 and preferably extending through sealant 34. Tubing 36 is provided with a stopcock or other suitable valve 38 proximate an end thereof. As shown in FIG. 3B, cover 30 next is disposed around instrument 32 and compressed onto mastic sealant 34 to create a seal between cover 30 and instrument 32. Finally, as shown in FIG. 3C, the fluid (gas/air) in region 30′ may be evacuated by attaching a pump to tubing 36. As the fluid is removed from region 30′, cover 30 contracts so that it more closely conforms to the shape of instrument 32. With a substantial amount of such fluid removed from within cover 30, as shown for example in FIG. 3C, the drape 30 surrounds instrument 32 to provide a desired form fit that may be optimal for the given medical procedure and handling of the instrument 32. As described previously, all fluid need not be evacuated from within cover 30, and the seal created by sealant 34 need only be sufficient for fluid to be evacuated from within cover 30 to permit the cover to conform to the shape of instrument 32 as needed.

While various descriptions of the present invention are described above, it should be understood that the various features can be used singly or in any combination thereof. Therefore, this invention is not to be limited to only the specifically preferred embodiments depicted herein.

Further, it should be understood that variations and modifications within the spirit and scope of the invention may occur to those skilled in the art to which the invention pertains. For example, cover 20, 30 may be formed of a bio-compatible polymer that may be subjected to heat to obtain further heat-shrink benefits. After gas has been evacuated from within cover 20, 30 to obtain general conformity between the shape of cover 20, 30 and the shape of instrument 22, 32, remaining excess material may be subjected to heat to obtain an even more conforming fit. Accordingly, all expedient modifications readily attainable by one versed in the art from the disclosure set forth herein that are within the scope and spirit of the present invention are to be included as further embodiments of the present invention. The scope of the present invention is accordingly defined as set forth in the appended claims.

1. A method for applying a medical instrument cover comprising:
providing a medical instrument comprising an elongate member, the elongate member having a first end configured for insertion into a body of a patient and a second end spaced from the first end;
providing a flexible cover having a closed end, an open end, and sidewall defining an inner region between the closed end and open end;
covering at least the first end of the elongate member with the flexible cover;
sealing the flexible cover and the instrument to each other to form an enclosed space comprising at least the first end of the elongate member;
evacuating fluid from within the enclosed space to form an evacuated enclosed space, with the flexible cover conforming to at least a portion of the elongate member within the evacuated enclosed space.

2. The method of claim 1, wherein the flexible cover and instrument are sealed together with mastic.

3. The method of claim 1, wherein the flexible cover and instrument are sealed together with an adhesive material disposed therebetween.

4. The method of claim 1, wherein the fluid is evacuated from the enclosed space so that the evacuated enclosed space has a substantially smaller volume than the enclosed space.

5. The method of claim 4, wherein the fluid is evacuated from the enclosed space so that the flexible cover substantially conforms to at least a portion of the elongate member within the evacuated enclosed space.

6. The method of claim 1, further comprising:
disposing a sealing material on the flexible cover within the inner region;
contacting the sealing material to the instrument to seal the flexible cover and the instrument to each other.

7. The method of claim 6, further comprising:
forming a discrete fluid path from the enclosed space to outside the flexible cover;
evacuating the fluid through the discrete fluid path to form the evacuated enclosed space.

8. The method of claim 7, wherein the discrete fluid path comprises a tube.

9. The method of claim 8, wherein the flexible cover, instrument and tube are sealed together with mastic sealing material.

10. The method of claim 9, wherein the tube extends through the mastic sealing material.

11. The method of claim 1, further comprising:
disposing a sealing material on the instrument;
contacting the sealing material to the flexible cover to seal the flexible cover and the instrument to each other.

12. The method of claim 11, further comprising:
forming a discrete fluid path from the enclosed space to outside the flexible cover;
evacuating the fluid through the discrete fluid path to form the evacuated enclosed space.

13. The method of claim 12, wherein the discrete fluid path comprises a tube.

14. The method of claim 13, wherein the flexible cover, instrument and tube are sealed together with mastic sealing material.

15. The method of claim 14, wherein the tube extends through the mastic sealing material.

16. The method of claim 1, wherein the flexible cover and the instrument are sealed to each other by applying an at least partially circumferential adhesive element nearer the second end of the elongate member than the first end.

17. The method of claim 1, wherein the evacuated enclosed space is not completely free of fluid.

18. The method of claim 1, wherein the flexible cover and instrument are sufficiently sealed to each other to form a substantially nonleaking union.

19. A method of forming a sterile field around a medical instrument comprising:
providing a medical instrument comprising an elongate member, the elongate member having a first end configured for insertion into a body of a patient and a second end spaced from the first end;
providing a flexible drape having a closed end, an open end, and a sidewall defining an inner region between the ends;
covering at least the first end of the elongate member with the flexible drape;
sealing the flexible drape and the instrument to each other with mastic to form a first enclosed space comprising at least the first end of the elongate member;
removing gas from within the first enclosed space to form a second enclosed space smaller than the first enclosed space, with the flexible drape conforming to at least a portion of the elongate member within the second enclosed space.

20. The method of claim 19, further comprising:
disposing the mastic on the flexible drape within the inner region;
contacting the mastic to the instrument to seal the flexible drape and the instrument to each other.

21. The method of claim 19, further comprising:
disposing the mastic on the instrument;
contacting the mastic to the flexible drape to seal the flexible drape and the instrument to each other.

22. The method of claim 19, further comprising:
forming a discrete fluid path from the first enclosed space to outside the flexible drape;
evacuating the gas through the discrete fluid path to form the second enclosed space.

23. The method of claim 19, wherein the gas is removed from the first enclosed space so that the second enclosed space has a substantially smaller volume than the first enclosed space.

24. The method of claim 23, wherein the flexible drape substantially conforms to at least a portion of the elongate member within the second enclosed space.

25. The method of claim 19, wherein the flexible drape and instrument are sufficiently sealed to each other to form a substantially nonleaking union.
